(19) United States
(12) Patent Application Publication
(10) Pub. No.: US 2024/0351027 A1
(43) Pub. Date: Oct. 24, 2024

(54) IMAGING DEVICE FOR AMI DIAGNOSIS

(71) Applicant: Wenzhou University, Zhejiang (CN)

(72) Inventors: Xiaojing Chen, Zhejiang (CN); Zhonghao Xie, Zhejiang (CN); Guangzao Huang, Zhejiang (CN); Yao Zhou, Zhejiang (CN); Qifan Chen, Zhejiang (CN); Lechao Zhang, Zhejiang (CN)

(73) Assignee: Wenzhou University, Zhejiang (CN)

(21) Appl. No.: 18/434,730

(22) Filed: Feb. 6, 2024

(30) Foreign Application Priority Data
Apr. 20, 2023 (CN) 202310438561.9

(51) Int. Cl. B01L 3/00

(52) U.S. Cl. CPC B01L 3/502715; B01L 2200/025; B01L 2200/16; B01L 2300/0654; B01L 2300/168

(57) ABSTRACT

Disclosed is an imaging device for AMI diagnosis includes a first polarizer is arranged in an imaging part and a second polarizer is arranged in a light source part. These two are linear polarizers, and the polarization directions of the first polarizer and the second polarizer are perpendicular to each other. During the imaging process, a near-infrared beam emitted from a near-infrared light source is modulated into a linearly polarized light with a certain angle when passing through the second polarizer. Then, it is irradiated onto a subject and the light is reflected due to the specular reflection effect of the subject turns into a cross-polarized light when passing through the first polarizer. The linearly polarized light perpendicular to the polarization angle of the first polarizer is intercepted, that is, the reflected light is intercepted.

### CROSS-REFERENCE TO RELATED APPLICATION

This application claims the priority benefit of Chinese application no. 202310438561.9, filed on Apr. 20, 2023. The entirety of the above-mentioned patent application is hereby incorporated by reference herein and made a part of this specification.

### BACKGROUND

### Technical Field

The invention relates to an imaging device, in particular to an imaging device for acute mesenteric ischemia (AMI) diagnosis.

### Description of Related Art

Acute mesenteric ischemia (AMI) refers to inadequate blood supply to the intestines caused by mesenteric artery embolism (50%), mesenteric artery thrombosis (15%-25%), mesenteric vein thrombosis (5%-15%) or other factors. Only about 1% of patients with acute abdominal diseases may be diagnosed with AMI, and for patients over 70 years old, the morbidity of AMI increases significantly.

The traditional AMI diagnosis method involves the comparison of circulating levels of glucagon-like peptide 2 (GLP-2) and/or glucagon-like peptide 1 (GLP-1) in a biological sample of a patient with those in a reference sample, as discussed in the Chinese Patent CN110036295A. This approach identifies and characterizes AMI by using the evaluation of GLP-2 and/or GLP-1. However, this method is not effective, as it cannot distinguish the specific position of mesenteric ischemia. Moreover, various serum markers similar to circulating glucagon-like peptides are considered as related to AMI, these parameters lack the required sensitivity and specificity for precise diagnosis of AMI.

To solve the limitations of traditional AMI diagnosis methods, an image-based AMI diagnosis method emerges. According to this method, a diphasic enhanced CT image is acquired and observed by professional doctors to draw a diagnostic conclusion. Although the image-based AMI diagnosis method can visibly show the intestinal condition of patients, the radiographic cost of patients is high due to the high price of ct equipment. Moreover, this method needs professional doctors for the whole diagnosis process, leading to a long diagnostic time and inconvenience.

At present, near-infrared reflectance spectroscopy (NIRS) and indocyanine green (ICG) have been applied to digital pathology, ophthalmology, gynecology and other systems by researchers, such as Chinse Patent Publication No. CN110582222A and Chinese Patent Publication No. CN210330538U. Moreover, there exists pertinent literature proving the practicality of the NIRS and ICG in mesenteric ischemia diagnosis, such as Barberio M, Felli E, Seyller E, et al. Quantitative fluorescence angiography versus hyperspectral imaging to assess bowel ischemia: A comparative study in enhanced reality [J]. Surgery, 2020, 168 (1): 178-184. The NIRS, as an emerging spectroscopic technique, can quickly and conveniently detect local tissue hypoxia. The ICG, as a near-infrared fluorochrome, can be excited by an extraneous light with a wavelength of 750-800 nm to emit a near-infrared light with a wavelength of about 830 nm. The variation in ICG concentration in the mesenteric vessel results in a weak signal response in a near-infrared fluorescent image. The part of mesenteric ischemia can be diagnosed by processing and analyzing the acquired near-infrared fluorescent image, without the experience and intuition of doctors. Therefore, researchers propose that NIRS can be used for AMI diagnosis. However, when NIRS is used for AMI diagnosis, some subtle features such as vascular patterns and surface irregularities are covered by glare points formed due to light reflection, leading to a low accuracy of the obtained near-infrared fluorescent image.

At present, there are many methods for avoiding glare spots. For example, in Patent CN204554597U and Patent CN115264425A, a dome (or tunnel) with a high-reflectivity coating is used, and the coating can diffusely reflect light from a light source to a sample. Through suitable design and installation measures, specular reflection glare from workpieces can be eliminated in the resulting image. However, due to the fact that this is an indirect lighting method, a high lighting power is needed to illuminate the dome or the inner surface of the tunnel to realize sufficient diffuse reflection irradiation of a sample to be scanned. Therefore, the diameter of the dome should be large enough to ensure diffusive illumination in the whole field of view of a camera. Although the accuracy of the near-infrared fluorescent image can be improved, the lighting equipment is heavy and expensive, also leading to a high radiographic cost of patients.

Another common method, as described in Patent CN114270395A, restrains glare by interpolation and estimation, as well as internal coating. Although this method do not lead to a high radiographic cost for patients, it cannot reconstruct the original information in the image, and the original information can only be estimated from the surroundings, which may lead to a loss of features in the glare area, resulting in an incorrect diagnosis.

### SUMMARY

The technical issue to be settled by the invention is to provide an imaging device for AMI diagnosis, which has a high diagnostic accuracy rate, can reduce the diagnostic cost and shorten the diagnostic time.

In brief, the imaging device for AMI diagnosis comprises an imaging part and a light source part, wherein the light source part is configured to excite ICG in the sample to produce a fluorescent light. The imaging part is configured to acquire a near-infrared image of the sample and comprises a near-infrared image acquisition mechanism and a first filter. The first filter is located in front of the near-infrared image acquisition mechanism. The light source part comprises a near-infrared light source and a second filter. The near-infrared light source is located in front of the first filter. The second filter is located in front of the near-infrared light source. The imaging part further comprises a first polarizer, which is located in front of the first filter and behind the near-infrared light source. The light source part further comprises a second polarizer, which is located in front of the second filter. The near-infrared image acquisition mechanism, the first filter, the first polarizer, the near-infrared light source, the second filter and the second polarizer are located on a same light path. Light holes are formed in a center of the near-infrared light source, a center of the second filter and a center of the second polarizer and communicated in a front-back direction. The fluorescent light produced by the sample enters the imaging part via the light holes. The first polarizer and the second polarizer are both linear polarizers, and the linear polarization directions of the first polarizer and the second polarizer are perpendicular to each other. The first filter is a bandpass filter and has a central wavelength of 832 nm and a bandwidth of 37 nm. The second filter is a bandpass filter or a short wave-pass filter. In a case where the second filter is the bandpass filter, the second filter has a central wavelength of 769 nm and a bandwidth of 41 nm. In a case where the second filter is a short wave-pass filter, the second filter has a cut-off wavelength of 800-810 nm. The near-infrared light source has a central wavelength of 780 nm. When an AMI diagnosis needs to be performed on a suspected AMI patient, an ICG saline solution with a concentration of 2.5-5 mg/ml is injected into the suspected AMI patient, the total amount of the ICG saline solution injected into the suspected AMI patient is determined according to the total weight of the suspected AMI patient. 0.5 mg of the ICG saline solution needs to be injected per kilogram. Then, the suspected AMI patient, as a subject, stands at a preset position in front of the second polarizer, at this moment, the subject is located at an imaging center of the near-infrared image acquisition mechanism. The near-infrared image acquisition mechanism and the near-infrared light source are switched on, the near-infrared light source emits a beam with a central wavelength of 780 nm to the second filter. The beam is filtered by the second filter to allow the near-infrared beam within the wavelength range thereof to be emitted out and transmitted to the second polarizer. The second polarizer allows the emission of a modulated beam in the linear polarization direction. Then, the modulated near-infrared beam is irradiated onto the sample. On one hand, ICG in the body of the sample is excited to produce the fluorescent light, and on the other hand, a reflected light is generated due to the specular reflection of the sample. The fluorescent light and the reflected light form a mixed light, which sequentially passes through the light holes in the center of the second polarizer, the center of the second filter and the center of the near-infrared light source is transmitted to the first polarizer. The first polarizer allows the light, in the linear polarization direction and the mixed light is passed through and irradiated onto the first filter, and is filtered by the first filter and allow light with a wavelength of 813-850 nm to be irradiated the near-infrared image acquisition mechanism through the first filter. The near-infrared image acquisition mechanism acquires a light signal, generates a fluorescent image, and displays the near-infrared fluorescent image.

The near-infrared light source has an illuminance of not less than 1000 lx.

The near-infrared light source has an illuminance greater than 1500 lx.

The near-infrared image acquisition mechanism is a near-infrared camera.

The first filter, the first polarizer and the near-infrared light source are all mounted on the near-infrared acquisition mechanism, and the second filter and the second polarizer are both mounted on the near-infrared light source.

Compared with the prior art, the invention has the following advantages. A first polarizer is arranged in an imaging part, a second polarizer is arranged in a light source part. The first polarizer and the second polarizer are both linear polarizers, the polarization directions of the first polarizer and the second polarizer are perpendicular to each other. The first filter is a bandpass filter and has a central wavelength of 832 nm and a bandwidth of 37 nm, and the second filter is a bandpass filter or a short wave-pass filter. In a case where the second filter is a bandpass filter, the second filter has a central wavelength of 769 nm and a bandwidth of 41 nm. In a case where the second filter is a short wave-pass filter, the second filter has a cut-off wavelength of 800-810 nm, and the near-infrared light source has a central wavelength of 780 nm. When an AMI diagnosis need to be performed for a suspected patient, an ICG saline solution with a concentration of 2.5-5 mg/ml is injected into the suspected AMI patient, the total amount of the ICG saline solution injected into the suspected AMI patient is determined according to the total weight of the suspected AMI patient, and 0.5 mg of the ICG saline solution needs to be injected per kilogram. Then, the suspected AMI patient, as a sample, stands at a preset position in front of the second polarizer, at this moment, the sample is located at an imaging center of the near-infrared image acquisition mechanism, the near-infrared image acquisition mechanism and the near infrared light source are switched on. The near infrared light source emits a near infrared beam with a central wavelength of 780 nm to the second filter, the near infrared beam is filtered by the second filter to allow the near-infrared beam within the wavelength range to be emitted out and transmitted to the second polarizer. The second polarizer allows a beam, in the linear polarization direction thereof, in the near-infrared beam transmitted thereto to be emitted out. The beam emitted from the second polarizer is a modulated near-infrared beam, which is irradiated onto the sample. On one hand, ICG in the body of the sample is excited to produce a fluorescent light, and on the other hand, a reflected light is generated due to specular reflection of the sample (because of the wet skin surface of the sample or other causes). The fluorescent light and the reflected light form a mixed light, which sequentially passes through light holes in a center of the second polarizer, a center of the second filter and a center of the near infrared light source is transmitted to the first polarizer. The first polarizer allows a light, in the linear polarization direction thereof, in the mixed light to pass through is irradiated onto the first filter. The first filter filters the light irradiated thereon to allow a light with a wavelength of 813-850 nm to pass through and irradiated onto the near infrared image acquisition mechanism. The near infrared image acquisition mechanism acquires a light signal, generates a near infrared fluorescent image, and outputs the near infrared fluorescent image. During the imaging process, the near-infrared beam emitted from the near-infrared light source is modulated by the second polarizer into a linearly polarized light with a certain angle when passing through the second polarizer. Then, irradiated onto the sample, the light reflected due to the specular reflection effect of the sample turns into a crossed polarized light when passing through the first polarizer. The linearly polarized light perpendicular to the polarization angle of the first polarizer is intercepted, that is, the reflected light is intercepted. In this way, glare spots produced by the property of the sample are filtered out, and a high-accuracy image is obtained. When the high-accuracy image is used for subsequent diagnosis, a high diagnostic accuracy rate can be realized, and the diagnostic time of patients can be shortened. In addition, the imaging device for AMI diagnosis is simple in structure, low in cost and has a low imaging cost, thus reducing the diagnostic cost of patients.

### BRIEF DESCRIPTION OF THE SEVERAL VIEWS OF DRAWINGS

FIG. 1 is a three-dimensional view of a light source part of an imaging device for AMI diagnosis according to the invention.

FIG. 2 is a sectional view of the light source part of the imaging device for AMI diagnosis according to the invention.

FIG. 3 is a three-dimensional view of an imaging part of the imaging device for AMI diagnosis according to the invention.

FIG. 4 is a sectional view of the imaging part of the imaging device for AMI diagnosis according to the invention.

FIG. 5 illustrates an imaging light path of the imaging device for AMI diagnosis according to the invention.

### DETAILED DESCRIPTION OF THE INVENTION

The invention will be described in further detail below in conjunction with accompanying drawings and embodiments.

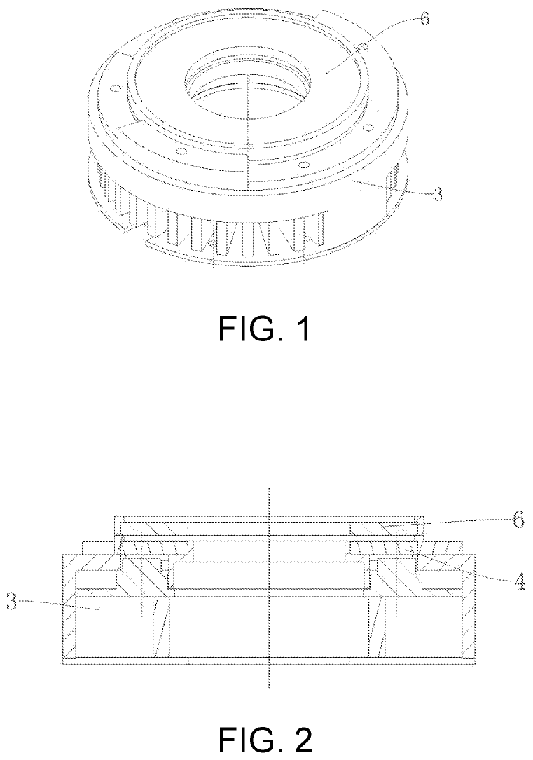

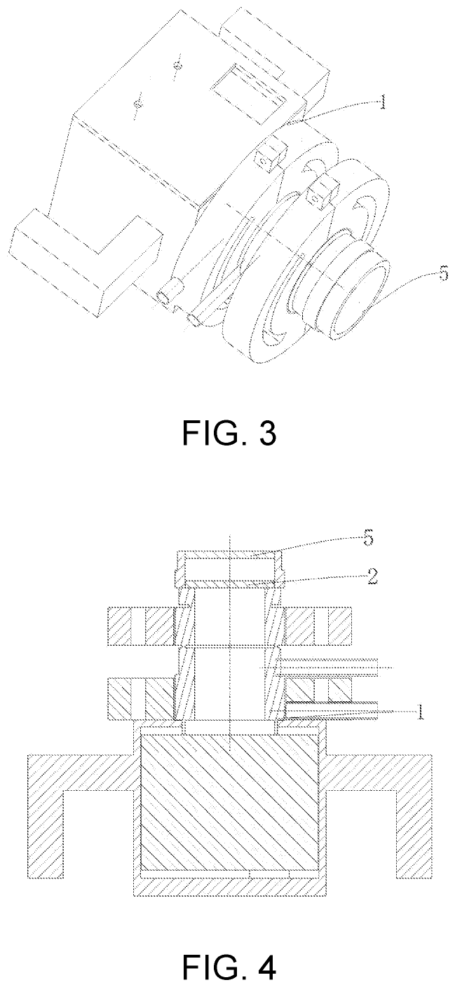

Embodiment 1: As shown in FIG. 1-FIG. 4, an imaging device for AMI diagnosis comprises an imaging part and a light source part, wherein the light source part is configured to excite ICG in the body of a sample to produce a fluorescent light. The imaging part is configured to acquire a near infrared image of the sample and comprises a near infrared image acquisition mechanism 1 and a first filter 2. The first filter 2 is located in front of the near infrared image acquisition mechanism 1, the light source part comprises a near infrared light source 3 and a second filter 4. The near infrared light source 3 is located in front of the first filter 2, the second filter 4 is located in front of the near infrared light source 3. The imaging part further comprises a first polarizer 5, which is located in front of the first filter 2 and behind the near infrared light source 3. The light source part further comprises a second polarizer 6, and the second polarizer 6 is located in front of the second filter 4. The near infrared image acquisition mechanism 1, the first filter 2, the first polarizer 5, the near infrared light source 3, the second filter 4 and the second polarizer 6 are located on a same light path. Light holes are formed in a center of the near-infrared light source 3, a center of the second filter 4 and a center of the second polarizer 6 are communicated in a front-back direction. The fluorescent light produced by the sample enters the imaging part via the light holes. The first polarizer 5 and the second polarizer 6 are both linear polarizers, and linear polarization directions of the first polarizer 5 and the second polarizer 6 are perpendicular to each other. The first filter 2 is a bandpass filter and has a central wavelength of 832 nm and a bandwidth of 37 nm. The second filter 4 is a bandpass filter or a short wave-pass filter. In a case where the second filter 4 is a bandpass filter, the second filter 4 has a central wavelength of 769 nm and a bandwidth of 41 nm. In a case where the second filter 4 is a short wave-pass filter, the second filter 4 has a cut-off wavelength of 800-810 nm. The near-infrared light source 3 has a central wavelength of 780 nm.

In this embodiment, the near-infrared light source 3 has an illuminance of not less than 1000 lx, and the near-infrared image acquisition mechanism 1 is a near-infrared camera.

Embodiment 2: This embodiment is basically the same as Embodiment 1, and differs from Embodiment 1 in that, in this embodiment, the near-infrared light source 3 has an illuminance greater than 1500 lx.

Embodiment 3: This embodiment is basically the same as Embodiment 1, and differs from Embodiment 1 in that, in this embodiment, the first filter 2, the first polarizer 5 and the near-infrared light source 3 are all mounted on the near-infrared acquisition mechanism 1, and the second filter 4 and the second polarizer 6 are both mounted on the near-infrared light source 3.

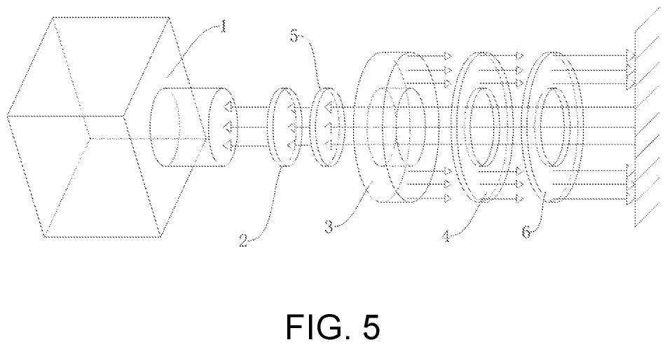

The imaging principle of the imaging device for AMI diagnosis provided by the invention is as follows. As shown in FIG. 5, when an AMI diagnosis needs to be performed on a suspected AMI patient, an ICG saline solution with a concentration of 2.5-5 mg/ml is injected into the suspected AMI patient, the total amount of the ICG saline solution injected into the suspected AMI patient is determined according to the total weight of the suspected AMI patient, and 0.5 mg of the ICG saline solution needs to be injected per kilogram. Then, the suspected AMI patient, as a sample, stands at a preset position in front of the second polarizer 6, at this moment, the sample is located at an imaging center of the near infrared image acquisition mechanism 1, the near infrared image acquisition mechanism 1 and the near infrared light source 3 are switched on. The near infrared light source 3 emits a near infrared beam with a central wavelength of 780 nm to the second filter 4. The second filter 4 filters the near infrared beam irradiated to allow the near-infrared beam within the wavelength range thereof to be emitted out to be transmitted to the second polarizer 6. The second polarizer 6 allows a beam, in the linear polarization direction thereof, in the near infrared beam transmitted thereto to be emitted out, and the beam emitted from the second polarizer 6 is a modulated near-infrared beam, which is irradiated onto the sample. On one hand, ICG in the body of the sample is excited to produce a fluorescent light, and on the other hand, a reflected light is generated due to the specular reflection of the sample. The fluorescent light and the reflected light form a mixed light, which sequentially passes through the light holes in the center of the second polarizer 6, the center of the second filter 4 and the center of the near-infrared light source 3 is transmitted to the first polarizer 5. The first polarizer 5 allows a light, in the linear polarization direction thereof, in the mixed light to pass through and irradiated onto the first filter 2. The first filter 2 filters the light irradiated thereon to allow light with a wavelength of 813-850 nm to pass through and irradiated onto the near-infrared image acquisition mechanism 1, and the near-infrared image acquisition mechanism acquires a light signal, generates a near-infrared fluorescent image, and outputs the near-infrared fluorescent image. During the imaging process, the near-infrared beam emitted from the near infrared light source 3 is modulated by the second polarizer 6 into a linearly polarized light with a certain angle when passing through the second polarizer 6 and is then irradiated onto the sample. Then the light reflected due to the specular reflection effect of the sample turns into a crossed polarized light when passing through the first polarizer 5. The linearly polarized light perpendicular to the polarization angle of the first polarizer 5 is intercepted, that is, the reflected light is intercepted. In this way, glare spots produced by the property of the sample are filtered out, and a high-accuracy image is obtained.

What is claimed is:

1. An imaging device for AMI diagnosis, comprising an imaging part and a light source part, wherein the light source part is configured to excite indocyanine green in a sample to produce a fluorescent light, the imaging part is configured to acquire a near-infrared image of the sample and comprises a near-infrared image acquisition mechanism and a first filter, the first filter is located in front of the near-infrared image acquisition mechanism, the light source part comprises a near-infrared light source and a second filter, the near-infrared light source is located in front of the first filter, the second filter is located in front of the near-infrared light source, the imaging part further comprises a first polarizer, which is located in front of the first filter and behind the near-infrared light source, the light source part further comprises a second polarizer, which is located in front of the second filter, the near-infrared image acquisition mechanism, the first filter, the first polarizer, the near-infrared light source, the second filter and the second polarizer are located on a same light path, light holes are formed in a center of the near-infrared light source, a center of the second filter and a center of the second polarizer and communicated in a front-back direction, the fluorescent light produced by the sample enters the imaging part via the light holes, the first polarizer and the second polarizer are both linear polarizers, and linear polarization directions of the first polarizer and the second polarizer are perpendicular to each other, the first filter is a bandpass filter and has a central wavelength of 832 nm and a bandwidth of 37 nm, the second filter is a bandpass filter or a short wave-pass filter, in a case where the second filter is the bandpass filter, the second filter has a central wavelength of 769 nm and a bandwidth of 41 nm; in a case where the second filter is a short wave-pass filter, the second filter has a cut-off wavelength of 800-810 nm; the near-infrared light source has a central wavelength of 780 nm, when the AMI diagnosis needs to be performed on a suspected AMI patient, an indocyanine green saline solution with a concentration of 2.5-5 mg/ml is injected into the suspected AMI patient, the total amount of the ICG saline solution injected into the suspected AMI patient is determined according to the total weight of the suspected AMI patient, 0.5 mg of the ICG saline solution needs to be injected per kilogram, then, the suspected AMI patient, as the sample, stands at a preset position in front of the second polarizer, at this moment, the sample is located at an imaging center of the near-infrared image acquisition mechanism, the near-infrared image acquisition mechanism and the near-infrared light source are switched on, the near-infrared light source emits a beam with a central wavelength of 780 nm to the second filter, the beam is filtered by the second filter to allow a near-infrared beam within the wavelength range thereof to be emitted out and transmitted to the second polarizer, the second polarizer allows the emission of a modulated beam in the linear polarization direction thereof, then, the modulated beam is irradiated onto the sample, on one hand, ICG in the body of the sample is excited to produce the fluorescent light, and on the other hand, a reflected light is generated due to the specular reflection of the sample, the fluorescent light and the reflected light form a mixed light, which sequentially passes through the light holes in the center of the second polarizer, the center of the second filter and the center of the near-infrared light source is transmitted to the first polarizer, the first polarizer allows the light, in the linear polarization direction and the mixed light is passed through and irradiated onto the first filter, a irradiated light is filtered and allow light with a wavelength of 813-850 nm to pass through the near-infrared image acquisition mechanism, the near-infrared image acquisition mechanism acquires a light signal, generates a fluorescent image, and displays the near-infrared fluorescent image.

2. The imaging device for AMI diagnosis according to claim 1, wherein the near-infrared light source has an illuminance of not less than 1000 lx.

3. The imaging device for AMI diagnosis according to claim 1, wherein the near-infrared light source has an illuminance greater than 1500 lx.

4. The imaging device for AMI diagnosis according to claim 1, wherein the near-infrared image acquisition mechanism is a near-infrared camera.

5. The imaging device for AMI diagnosis according to claim 1, wherein the first filter, the first polarizer and the near-infrared light source are all mounted on the near-infrared acquisition mechanism, and the second filter and the second polarizer are both mounted on the near-infrared light source.
